(19) United States
(12) Patent Application Publication
(10) Pub. No.: US 2008/0215096 A1
(43) Pub. Date: Sep. 4, 2008

(54) Devices to Stabilise the Lamina

(76) Inventors: Lance Nash, Dunedin (NZ); Stephen Farquhar, Dunedin (NZ); Richard Cathro, Dunedin (NZ); John Galvin, Dunedin (NZ)

Correspondence Address: BLAKELY SOKOLOFF TAYLOR & ZAFMAN LLP, 1279 OAKMEAD PARKWAY, SUNNYVALE, CA, 94085-4040, US

(21) Appl. No.: 11/547,992

(22) PCT Filed: Apr. 7, 2005
(86) PCT No.: PCT/NZ05/00070

§ 371 Date: Nov. 30, 2007

(30) Foreign Application Priority Data
Apr. 7, 2004 (NZ) 528688

(51) Int. Cl. A61B 17/70

(52) U.S. Cl. 606/249

(57) ABSTRACT

A surgical implant device for stabilising a lamina of the spine following a procedure in which a cut is made in the lamina to form two lamina portions. The device has a longitudinally extending spacer component 211 adapted to extend across the cut between the lamina portions and adapted to be fixed to the lamina. The device may have one or more members 215 extending from one side of the spacer component to fit in the space between the two portions of the cut lamina when the device is fixed in position. The device may comprise a first spacer part and a second spacer part which are adapted to be positioned on opposite sides of the cut and connectable together to fix the device in position.

### FIELD OF INVENTION

This invention comprises devices that stabilise the lamina of the spine after a laminoplasty or other procedure in which a cut is made in the lamina to form two lamina portions.

### BACKGROUND

Cervical spinal stenosis is a condition in which the opening for the spinal cord that runs through the vertebrae of the spinal column is not quite wide enough to comfortably accommodate the spinal cord. The result is that when the ligaments thicken or there is some bulging of the cervical discs (a natural product of wear and tear through life) the person who is affected begins to experience pressure on their spinal cord. This causes pain in the head, neck and arms; weakness and/or numbness and tingling in the arms; and/or clumsiness in the arms and legs. As a result of these symptoms, some people find that their mobility and their ability to undertake a normal day's work is severely affected.

In a laminoplasty procedure the back of the spine is exposed, and the affected lamina are cut and bent outwards opening the spinal canal and providing more room for the spinal cord. The problem has then been to stabilise the lamina in this new position.

One way of stabilising the lamina is to take a bone graft from the hip in the form of a rectangular plate of bone and wedge it in position to try and hold the lamina in its new, more open shape. This is generally effective but because it is not a firm arrangement can lead to some slippage and recurrent narrowing of the spinal canal. It also involves making a separate wound in the area of the hip and taking a bone graft.

U.S. Pat. No. 6,080,157 discloses a prosthesis device to dynamically stabilise the lamina after laminoplasty.

Other medical devices are described in U.S. Pat. Nos. 5,496,318; 5,413,576; U.S. Pat. No. 5,282,863; and U.S. Pat. No. 4,604,995.

It is an object of the present invention to provide an alternative device to stabilise the lamina of the spine.

### SUMMARY OF INVENTION

The term ‘comprising’ as used in this specification and claims means ‘consisting at least in part of’, that is to say when interpreting statements in this specification and claims which include that term, the features prefaced by that term in each statement all need to be present but other features can also be present.

In accordance with a first aspect of the present invention, there is provided a surgical implant device for stabilising a lamina of the spine after a laminoplasty in which a cut is made in the lamina to form two lamina portions, the device comprising a longitudinally extending spacer component adapted to be fixed to the lamina across the cut, and comprising one or more members extending from one side of the spacer component to fit in the space between the two lamina portions when the device is fixed in position.

The spacer component may be contoured to substantially conform to the surface shapes of the lamina portions.

The spacer component may have an enlarged end configured to engage a portion of the lamina in use to distribute load. The spacer component may be generally L-shaped.

Preferably, the or each member which extends from one side of the spacer component comprises one or more apertures, to enhance bone growth and assist in fusing of the lamina of the device.

The or each member which extends from one side of the spacer body may comprise two spaced apart tabs, against which the cut surfaces of the lamina can abut in use. In an alternative form the member may comprise a single spacer block provided on one side of the spacer body.

The device is preferably configured to be fixed to both lamina portions.

The device may be configured to be fixed to the or each lamina portion by one or more fasteners such as screws passing through aperture(s) provided in the spacer component and into the or each lamina portion. The device is preferably configured such that the head(s) of the fasteners is/are accessible from the opposite side of the spacer component to that which engages the lamina portions.

The device preferably comprises a tab associated with the or each aperture which can be bent over the head of a respective fastener once tightened, to assist in maintaining the fastener in the tightened position.

In accordance with a second aspect of the present invention, there is provided a surgical implant device for stabilising a lamina of the spine after a laminoplasty in which a cut is made in the lamina to form two lamina portions, the device comprising a longitudinally extending spacer component adapted to be fixed to the lamina across the cut, the spacer component being contoured to conform substantially to the surface shapes of the lamina portions, and comprising one or more members extending from one side of the spacer component to fit in the space between the two lamina portions when the device is fixed in position and which comprise apertures to assist in bone growth and thereby fusing of the lamina to the device.

In accordance with a third aspect of the present invention, there is provided a surgical implant device for stabilizing a lamina of the spine after a procedure in which a cut is made in the lamina to form two lamina portions, the device comprising first and second spacer parts adapted to be positioned on opposite sides of the cut and connectable together to fix the device in position.

Preferably, the first spacer part comprises a body which is arranged to extend across the cut formed in the lamina. The body may be contoured to conform substantially to the surface shapes of the lamina portions on the outside of the lamina.

The body may have an enlarged end configured to engage a portion of the lamina in use to distribute load. The body may be generally L-shaped.

Preferably, the second spacer part comprises a body which is arranged to extend across the cut formed in the lamina. The body of the second spacer part may be contoured to conform substantially to the surface shapes of the lamina portions on the inside of the lamina.

Preferably, at least one of the spacer parts comprises a portion arranged to bite into the bone material of the lamina to assist in securely locating the device relative to the lamina portions. Both spacer parts may comprise spikes depending therefrom which are arranged to bite into the bone material of the lamina to assist in fusing of the lamina to the device. The spikes are preferably positioned at or adjacent the ends of the spacer parts.

At least one of the spacer parts may comprise one or more members extending from one side of the spacer part to fit in the space between the two parts of the cut lamina when the device is fixed in position.

In a preferred form the two spacer parts are configured to be connected together by a fastener extending from one spacer part and attaching to the other spacer part, such as a threaded fastener which extends from one spacer part and attached to the other spacer part. The device is preferably configured such that a head of the fastener is accessible from the opposite side of the first spacer part to that which engages the lamina portions.

The device preferably comprises a tab associated with the aperture which can be bent over the head of the fastener once tightened, to assist in maintaining the fastener in the tightened position.

The lamina may be in the cervical region of the spine. Alternatively, the lamina may be in the lumbar region of the spine or the thoracic region of the spine.

In accordance with a fourth aspect of the present invention, there is provided a surgical implant device for stabilizing a lamina of the spine after a laminoplasty in which a cut is made in the lamina to form two lamina portions, the device comprising first and second spacer parts adapted to be positioned on opposite sides of the cut and arranged to extend across the cut from the first lamina portion to the second lamina portion and connectable together to fix the device in position, each spacer part being contoured to conform substantially to the surface shapes of the lamina portions, and each spacer part being provided with dependent spikes which are configured to bite into the surfaces of the lamina portions in use to assist in fusing of the lamina portions to the device.

In accordance with a fifth aspect of the present invention, there is provided a method of stabilising a lamina of the spine after a procedure in which a cut is made in the lamina to form two lamina portions, comprising the steps of positioning and fixing a surgical implant device as claimed in any one of the preceding claims to the lamina portions across the cut in the lamina.

Preferably, the surgical implant device is positioned such that part of the device extends between the lamina portions to maintain the lamina portions in a spaced apart configuration.

Preferably, the lamina is in the cervical region of the spine. The procedure may be a laminoplasty.

Alternatively, the lamina is in the thoracic or lumbar region of the spine.

This invention also consists in a method of stabilising reshaped lamina after a laminoplasty comprising the steps of positioning and fixing a spacer implant device as defined above between the severed ends of the lamina.

### BRIEF DESCRIPTION OF THE DRAWINGS

Preferred forms of the invention are described with reference to the accompanying drawings, in which:

FIG. 1 is a cross section through the back bone;

FIG. 2 is a rear view of a section of the back bone;

FIG. 3 is a section of the back bone with modifications made;

FIG. 4 is a cross section of the back bone illustrating the modifications made in a laminoplasty;

FIGS. 5 and 6 show a single vertebrae after laminoplasty with one preferred form of a device according to the invention fitted in place;

FIG. 7 is a perspective view of the first preferred form device;

FIG. 8 is a view, generally from the side, of a first part of a modified version of the device of FIGS. 5 to 7;

FIG. 9 is an underside perspective view of the part of FIG. 8;

FIG. 10 is an underside view of the part of FIG. 8;

FIG. 11 is a view, generally from the side, of a second part of a modified version of the device of FIGS. 5 to 7;

FIG. 12 is an overhead perspective view of the part of FIG. 11;

FIG. 13 is a perspective view showing the components of FIGS. 8 to 12 being brought into engagement;

FIG. 14 is a perspective view showing the components of FIGS. 8 to 12 when fully engaged;

FIGS. 15a, 15b, 15c, and 15d are top, side, perspective, and end views of an alternative first part for use in the device of FIGS. 13 and 14;

FIG. 15e is a perspective view of detail E of FIG. 15c;

FIGS. 16 and 17 are perspective views of a second preferred form device fitted to a vertebrae after laminoplasty;

FIG. 18 is a perspective view of the second preferred form device;

FIGS. 19a, 19b, 19c, and 19d are top, side, end, and perspective views respectively of a modified version of the second preferred form device;

FIGS. 20a, 20b, 20c, and 20d are top, side, end, and perspective views respectively of a further modified version of the second preferred form device;

FIGS. 21a, 21b, 21c, and 21d are top, side, perspective, and end views of the embodiment of a further modified version of the second preferred form device; and

FIG. 21e is a perspective view of detail E of FIG. 21a.

### DESCRIPTION OF PREFERRED EMBODIMENTS

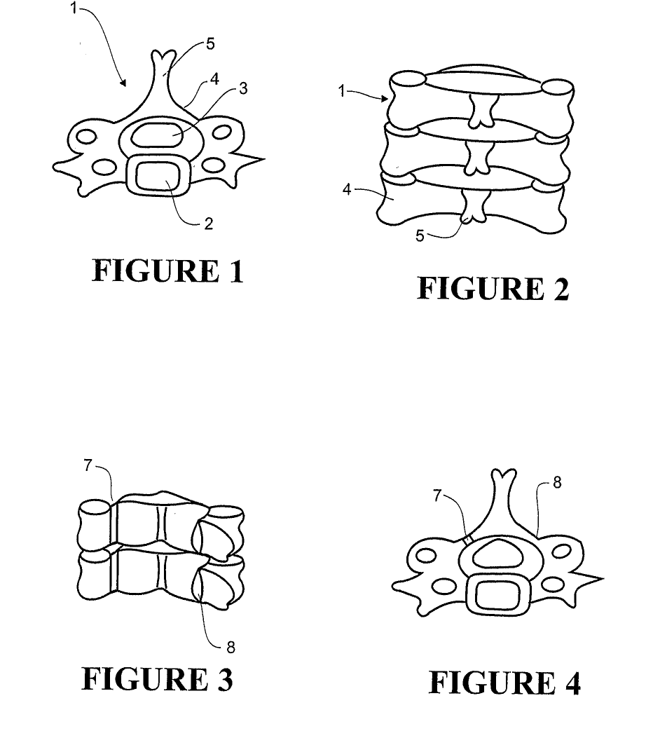

FIGS. 1 and 2 illustrate a section through a joint in the back bone 1 with the vertebrae body 2, spinal cord 3, lamina 4 and spine 5. FIG. 2 shows a rear view of a number of vertebrae.

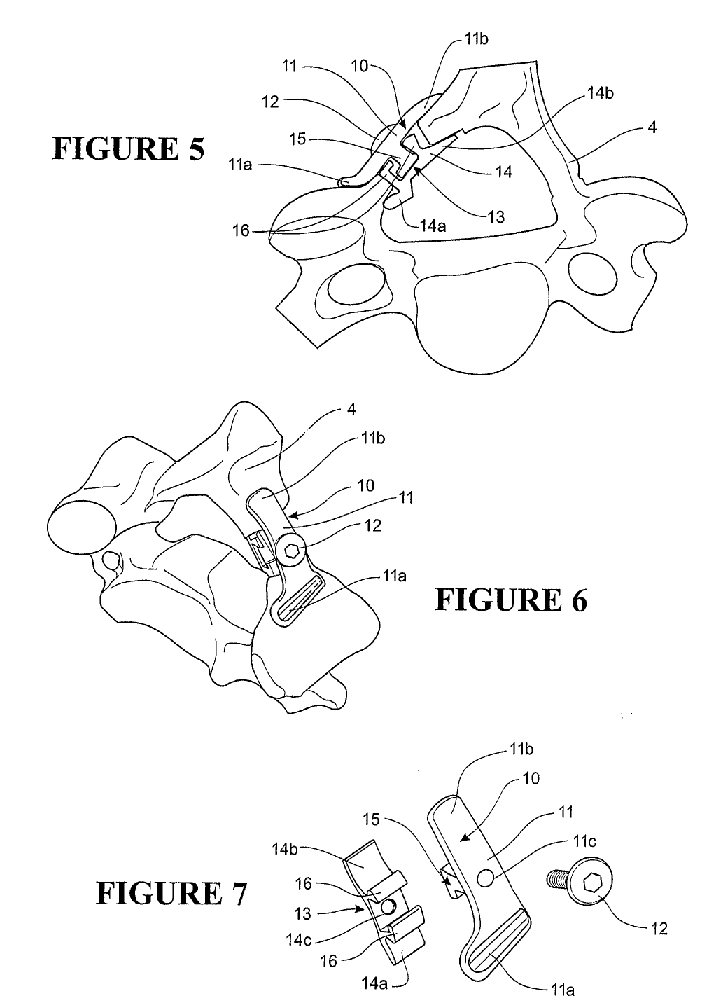

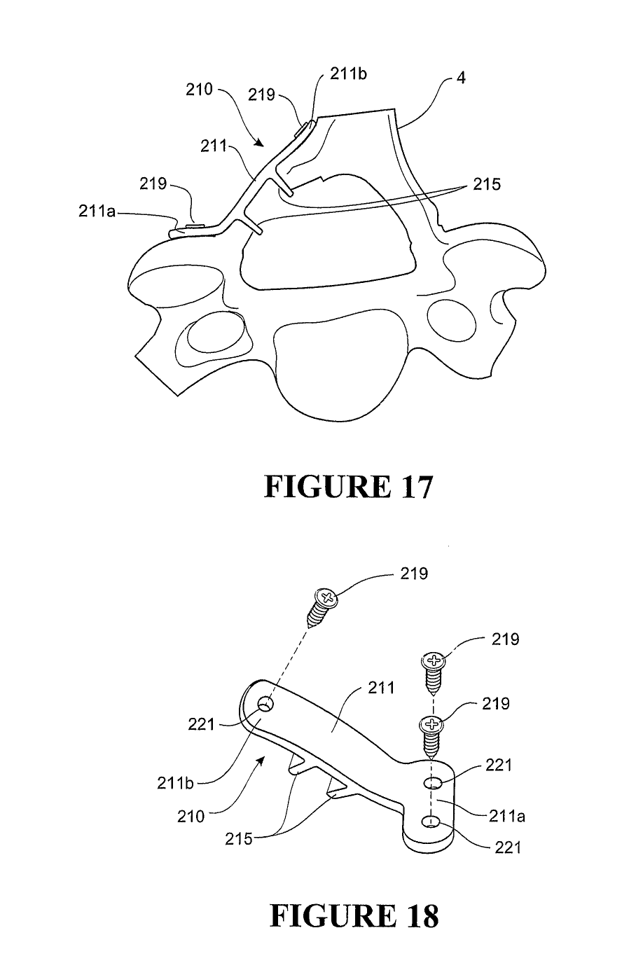

In a laminoplasty a cut 7 is made through the lamina on one side and a weakening groove 8 is formed on the other side (see FIGS. 3 and 4). This weakens the bone so that it can be deformed to a position as illustrated in FIGS. 5, 6, 16, and 17 to open the lamina and relieve pressure on the spinal cord, and allow insertion of a device to stabilise the deformed lamina in this opened position.

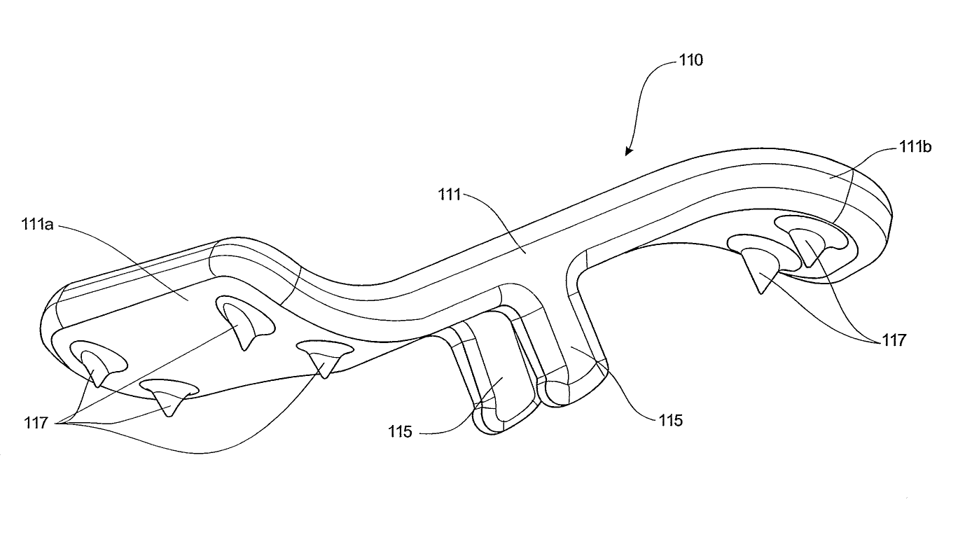

Referring to FIGS. 5 to 7, the first preferred form device for stabilising the cut lamina consists of two parts 10 and 13 which are fitted to an individual lamina as shown in FIGS. 5 and 6, and connected together by fastener 12 such as a screw which passes through part 10 and screws into part 13 to clamp the two parts of the device on either side of the cut lamina as shown. Alternatively the two parts 10 and 13 may tightly snap lock together for example by a bayonet moulding on one part engaging between a series of teeth on the other part.

In the form shown the part 10 includes a longitudinally extending body 11 which is arranged to extend across the cut in the lamina on the outside of the lamina from one portion of the cut lamina to the other portion of the cut lamina, and a member in the form of a leg 15 which extends from the body and is arranged to extend into the cut of the lamina. In the form shown the part 13 includes a longitudinally extending body 14 which is arranged to extend across the cut in the lamina on the inside of the lamina from one portion of the cut lamina to the other portion of the cut lamina, and two spaced wall parts 16 which extend from the body 14 of the part 13 as shown and are arranged to extend into the cut of the lamina. The leg 15 is arranged to engage in a channel between the two spaced wall parts 16. The two spaced wall parts 16 provide abutment surfaces against which the cut surfaces of the lamina can engage. Preferably the leg 15 is of an L-shape as shown and will engage under a laterally enlarged end of one or other of the wall parts 16. Alternatively the leg 15 or equivalent may project from part 13 towards the part 10 and the wall parts 16 from the part 10. Alternatively again either the leg 15 may be omitted and the fastener 12 may simply pass from the part 10 between the two wall parts 16 and screw into the body of the part 13, or the parts 16 may be omitted.

Preferably as shown one end 11a of the body 11 of part 10 is enlarged in a general shoe shape, such that body part 11 is substantially L-shaped. The shoe shape 11a is arranged to distribute load to the respective part of the lamina.

The undersides of the ends 11a, 11b of the part 10 and the undersides of the ends 14a, 14b of part 13 may also comprise a roughened gripping surface which will bite into the bone material of the cut lamina to assist in securely locating the implant device.

Each of parts 10, 13 are preferably contoured to conform substantially to the surfaces of the portions of the lamina on either side of the cut. In particular, the body 11 preferably has a generally planar central portion, a first curved portion 11a configured to conform substantially to the shape of the outer surface of one portion of the cut lamina, and a second curved portion 11b curved in the opposite direction to conform substantially to the shape of the outer surface of the other portion of the cut lamina. Similarly, the body 14 of the second part 13 preferably has a generally planar central portion, a first curved portion 14a configured to conform substantially to the shape of the inner surface of one part of the cut lamina, and a second portion 14b configured to conform substantially to the shape of the inner surface of the other part of the cut lamina. The shapes are shown most clearly in FIG. 5.

To fasten the device across the cut of the lamina, the second part 13 is inserted through the cut in the lamina on an orientation generally transverse to that shown in FIG. 5 to be positioned inside the vertebrae. The second part 13 is then turned so that the body 14 bridges across the cut from one portion of the cut lamina to the other portion of the cut lamina, and the fastener 12 which extends through an aperture 11c in the body 11 and into threaded aperture 14c of part 13 is tightened to tighten the parts 10, 13 together and generally sandwich the portions of the cut lamina therebetween to stabilise the lamina.

The device is suitably configured so that the fastener can be inserted though part 10 from the side opposite to the side which contacts the lamina parts. That is, the aperture 11c is exposed on the outer surface of the part 10 so that the head of the fastener 12 can be accessed from outside the lamina. The following devices are also preferably configured so that the fasteners are externally accessible.

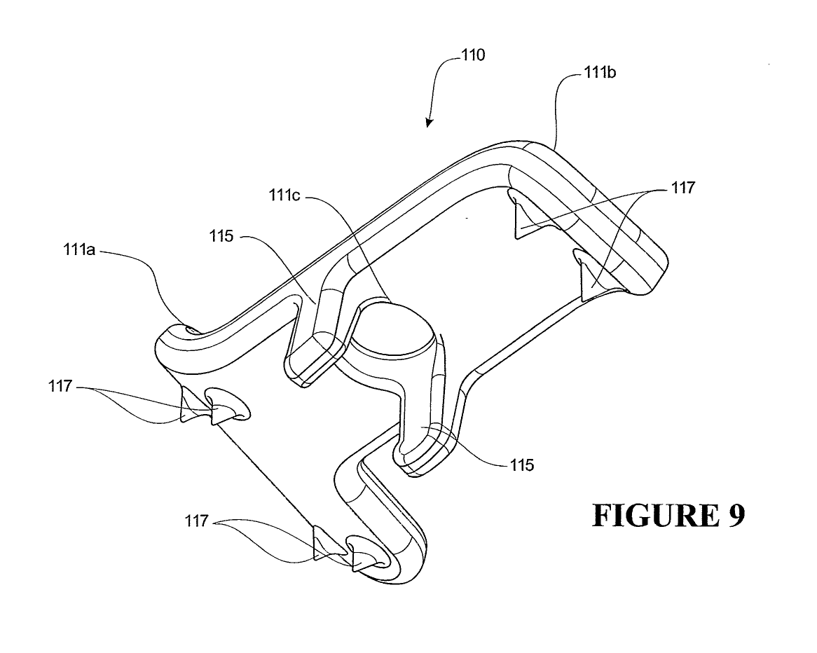

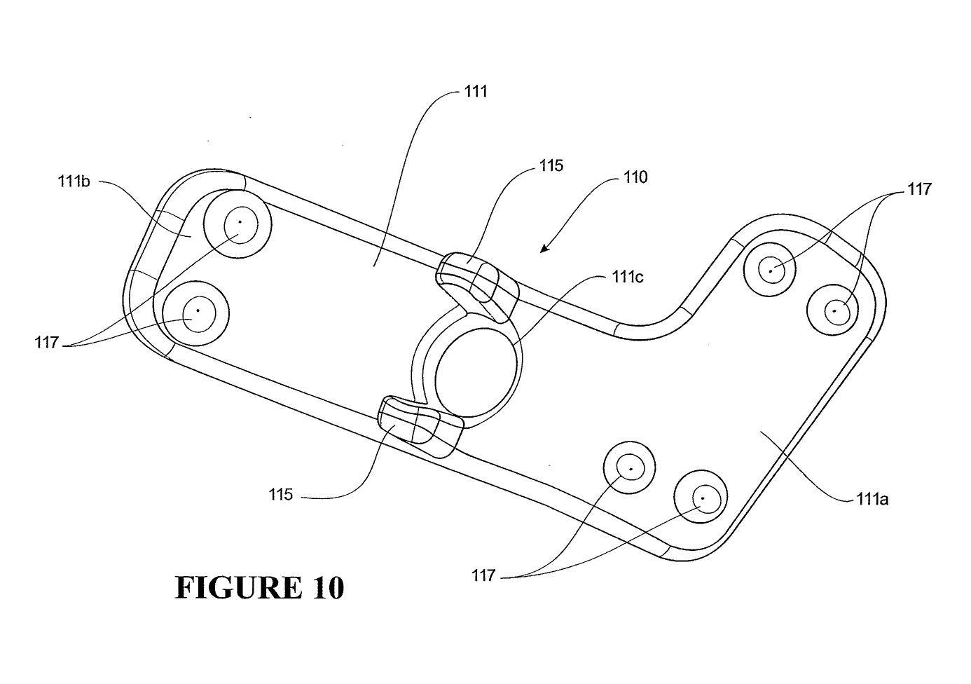

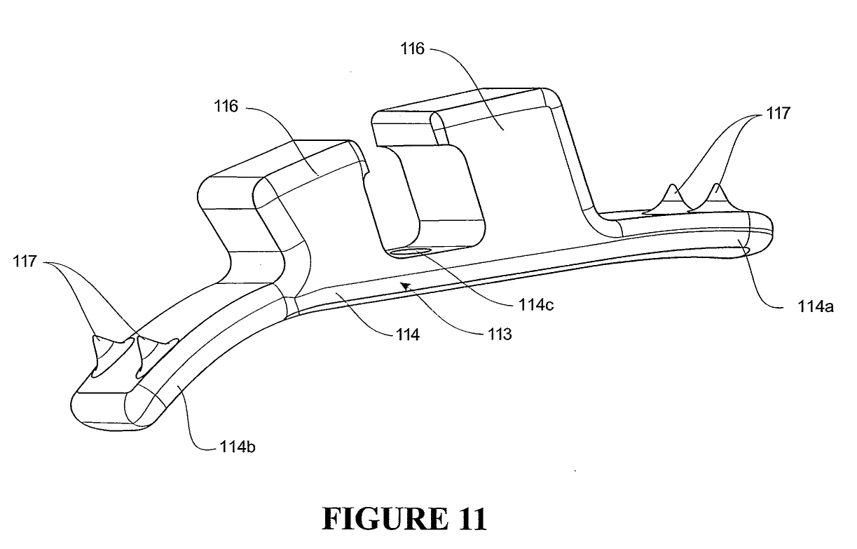

FIGS. 8 to 12 show a modified form of the device of FIGS. 5 to 7, which comprises a first part 110 shown in FIGS. 8, 9, and 10, and a second part 113 shown in FIGS. 11 and 12. Unless described below, the features and operation should be considered the same as the embodiment described with reference to FIGS. 5 to 7, and like reference numerals are used to indicate like parts, with the addition of 100.

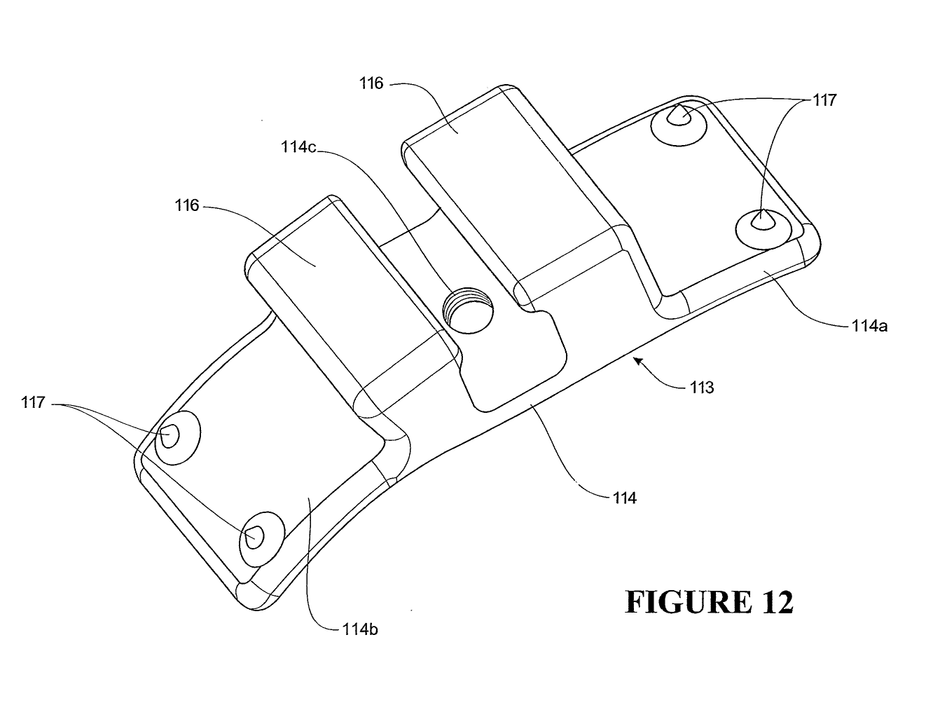

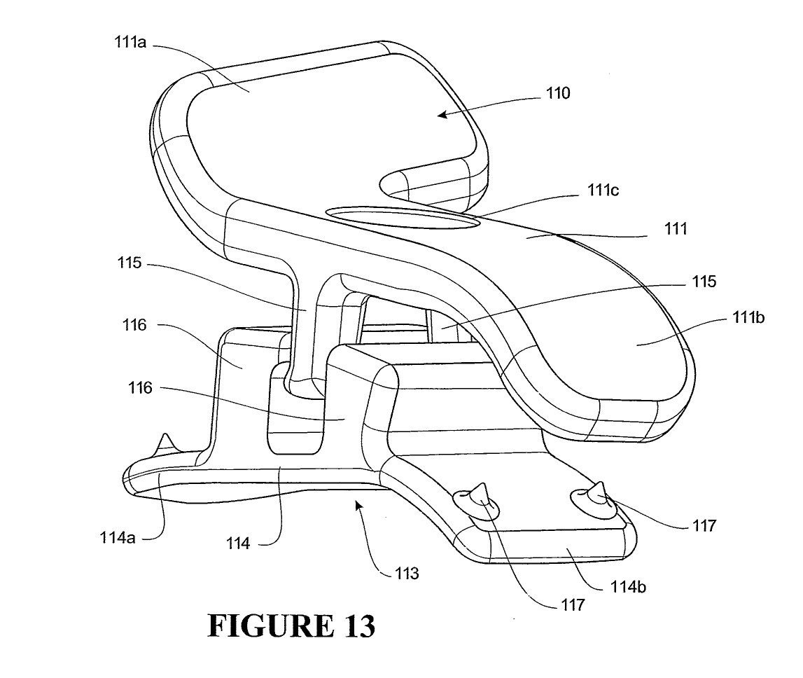

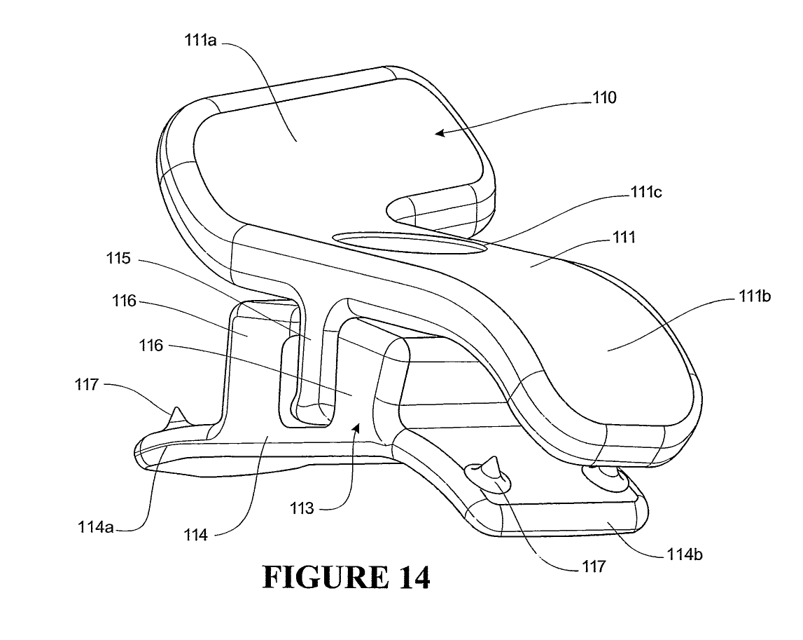

In this form, two generally L-shaped legs 115 extend from the body 111 of the first part 110, one on either side of the aperture 111c for receipt of a fastener (not shown). When the parts 111, 113 are connected together, the ends of the members 115 extend between the members 116 of the second part 113. The members 116 provide surfaces against which the cut edges of the lamina may contact, retaining the cut edges in a desired spaced apart configuration. It should be noted that the part 113 is shown in FIGS. 11 and 12 in the opposite orientation to that in which it would be connected to member 110 in use. That is, in use, part 114a of the body 114 would be generally aligned with part 111a of the body 111, and part 114b of the body 114 would be generally aligned with part 111b of body 111 as shown in FIGS. 13 and 14.

At least the end portions of parts 110, 113 preferably include spikes 117 which extend from the respective body parts. In use, as the parts 110, 113 are pulled towards one another as a result of tightening the fastener which extends between the parts, the spikes bite into the respective surfaces of the lamina. In use, over time stem cells will form around the spikes, and calcification will occur. That will assist in the bone fusing to the parts 110, 113 of the device, providing additional stability to the lamina. As can be seen in the Figures, the enlarged region 111a of the body 111 preferably includes a greater number of spikes (such as four as shown for example) than the opposite end portion 111b of the body 111 (which may have two as shown for example). That assists in distributing load to the portion of the cut lamina corresponding to the enlarged region 111a of the body 111.

FIG. 13 shows the parts 110, 113 being brought into engagement, and FIG. 14 shows the parts 110, 113 when fully engaged. In the position shown in FIG. 14, the lamina parts will be captured between parts 110 and 113.

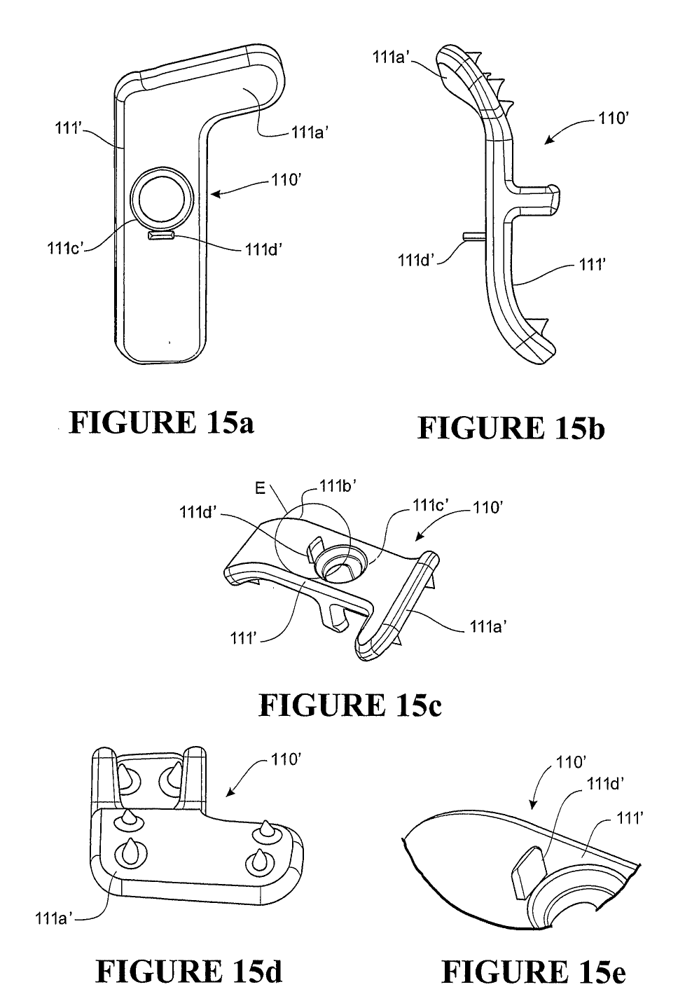

FIGS. 15a-15e show a modified first part for use in the device of FIGS. 13 and 14. Unless described below, the features and operation should be considered the same as the first part of FIGS. 8 to 10, and like reference numerals are used to indicate like parts with the addition of a prime (′). This part differs in that a tab 111d′ is provided adjacent the aperture 111c′, extending from the surface of the part 110′ opposite that which engages the lamina. Once this first part 110′ has been fastened to the second part 113 with a fastener extending through the aperture 111c′ and into aperture 114c and the fastener has been tightened, the tab 111d′ is bent over the aperture to at least partly cover the head of the fastener to assist in maintaining the fastener in the tightened position.

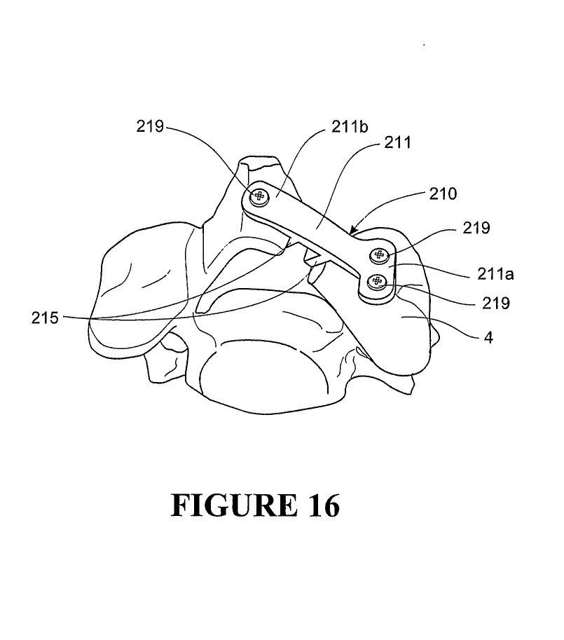

The second preferred form device 210 of FIGS. 16 to 18 is similar in shape to the that of FIGS. 5 to 7 except that the part 13 and fastener 12 which enable the device to be clamped in position as previously described are omitted, and the device is secured in position by fasteners such as screws 219 which pass through apertures 221 in the body 211 of the device 210 and into the parts of the cut lamina. The device 210 is generally similar in shape to part 10 of the device of FIGS. 5 to 7, except that at least one member, and in the embodiment shown two tabs 215, extend from the underside of the body 211 of the device as shown. In fitting of the device the tabs of the device are inserted between the cut ends of a lamina such that the cut surfaces of the lamina can abut the tabs 215, which serves to space the cut ends of the lamina appropriately.

Again, the device is preferably contoured as shown to conform substantially to the surfaces of the lamina portions. Again, an enlarged body portion 211a is preferably provided to distribute load in a desirable manner to the lamina. In the embodiment shown, the portion 211a is provided with a greater number of apertures 221 for receipt of fasteners than the opposite end of the body portion 211, to assist in the load distribution.

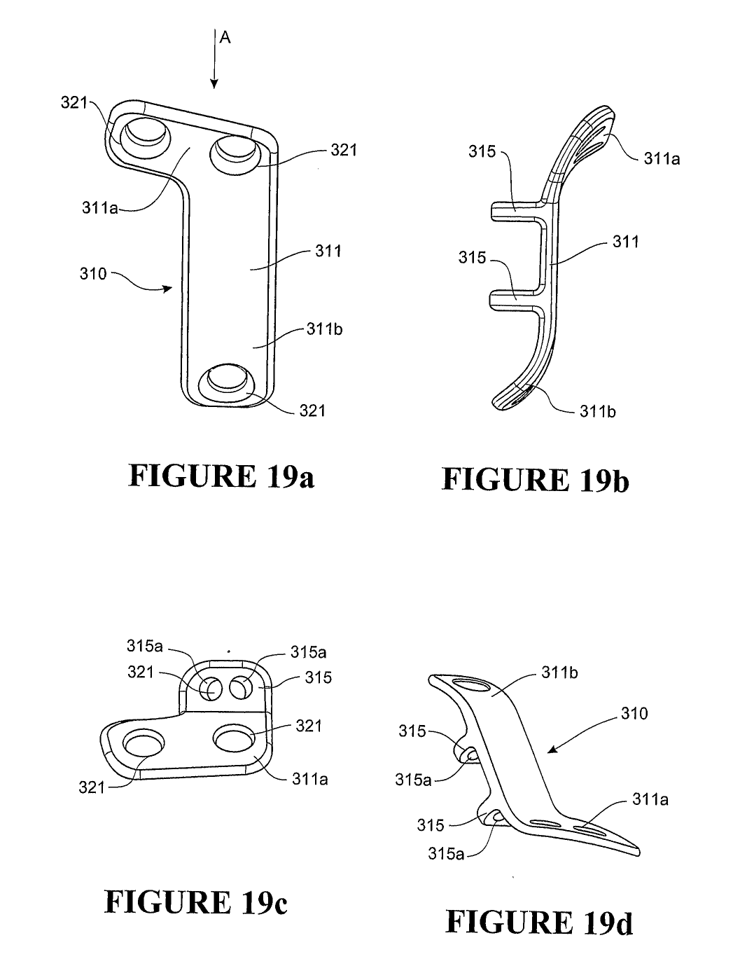

The embodiment of FIGS. 16 to 18 is configured for fastening to a cut lamina on the left side of the spine when viewed from the front. FIGS. 19a to 19d show an example 310 which is configured for fastening to a cut lamina on the right side of the spine when viewed from the front. Unless described below, the features and operation should be considered the same as for the embodiment of FIGS. 16 to 18, and like reference numerals are used to indicate like parts with the addition of 100. As can be seen, the enlarged portion 311a extends from the opposite side of the body 311 to the embodiment of FIGS. 16 to 18. This embodiment also differs in that the ledges are provided with apertures 315a, as can be seen from FIG. 19c which is a view in the direction of arrow A in FIG. 19a. These apertures 315a promote bone growth from the cut surfaces of the lamina, and will improve the fusing of the cut surfaces of the lamina to the device to enhance the stabilising of the lamina. Similar apertures could be formed in the ledges 214 of the embodiment of FIGS. 16 to 18.

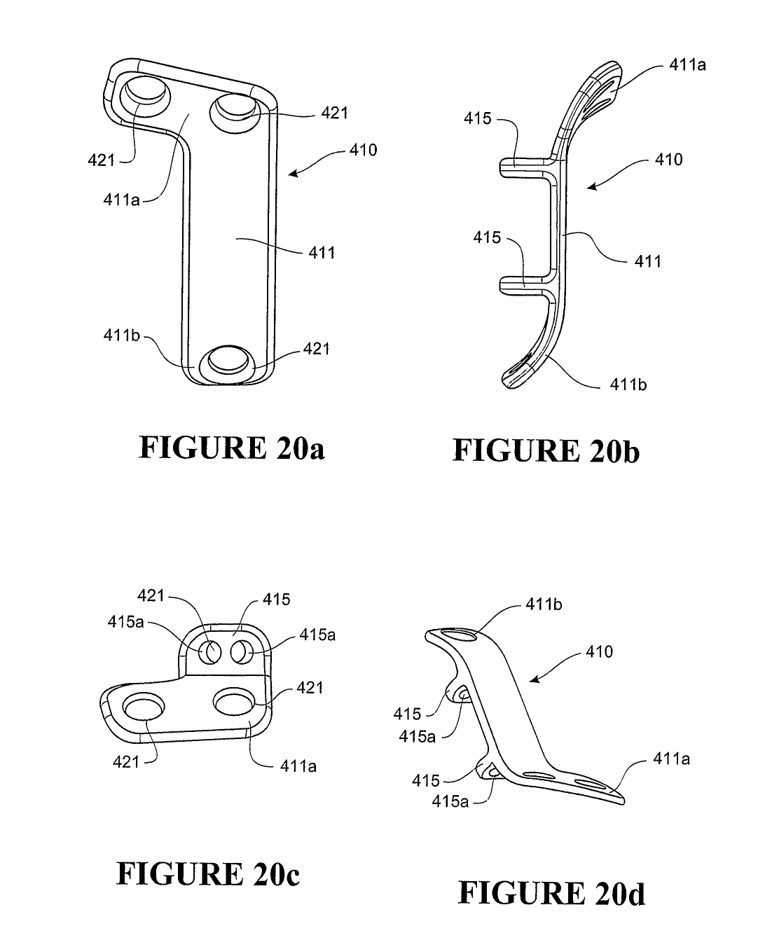

A further example 410 which is configured for attachment to a cut lamina on the right side of the spine (from the front) is shown in FIGS. 20a to 20d. Unless described below, the features and operation should be considered the same as for the embodiment of FIGS. 19a to 19d, and like reference numerals indicate like parts with the addition of 100. As can be seen in particular from FIG. 20b, the generally planar central portion of the body 411 is longer than in the example of FIG. 19b. Also, there are differences in the depth and curvature of the portions 411a, 411b of the body, over that of FIG. 19b. The applicant has determined that by providing nine variants for right side fixation and nine variants for left side fixation, it is possible to provide devices which will be contoured to suit the curvature in the laminal arch for 80% of patients, for the main cervical vertebral groups in which laminoplasty would typically be undertaken; those being C3/C4, C5/C6, and C7.

Again, the ends of these devices could be provided with roughened gripping surfaces or similar to bite into the bone material.

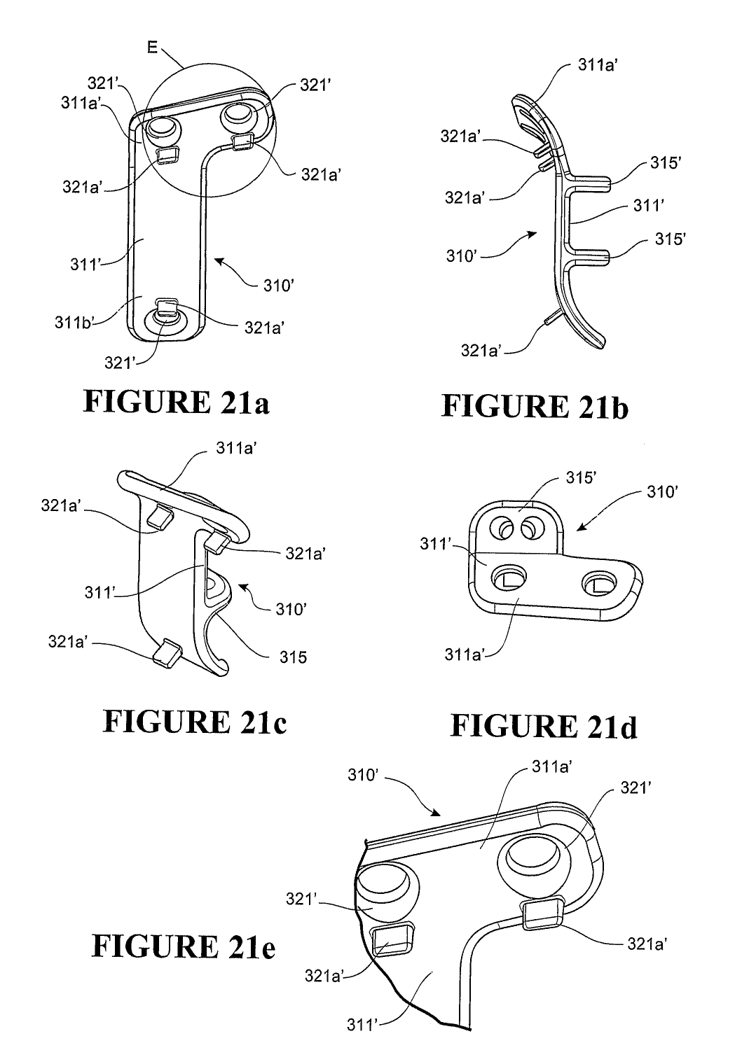

FIGS. 21a-21e show a further modified second embodiment device. Unless described below, the features and operation should be considered the same as the device of FIGS. 19a to 19d, and like reference numerals are used to indicate like parts with the addition of a prime (′). This part differs in that a tab 321a′ is provided adjacent each aperture 321′, extending from the surface of the part 310′ opposite that which engages the lamina. Once this part 310′ has been fastened to the lamina with fasteners extending through the apertures 321′ and the fastener has been tightened, the tabs 321a′ are bent over the apertures to at least partly cover the head of the fasteners to assist in maintaining the fasteners in the tightened positions.

The devices can be made from stainless steel or any other suitable material which will not be rejected by the body. The devices may be made from nylon, Teflon and/or titanium which will be compatible with the MRI scans should such scans need to be done following surgery, or alternatively from a ceramics material.

Use of devices of the present invention enable a reduction in operating time, and to lessen the theoretical likelihood of recurrent narrowing of the spine. They provide relatively rigid stabilising of the cut lamina. Another significant advantage for the patient is a more comfortable resulting neck than is achieved using other known procedures. As a consequential advantage over the need for removing a bone graft from the hip, the patient will have no discomfort in the hip which is often the most painful part of the traditional laminoplasty method.

The technique applied in fitting the device uses known means to encourage fusion between the device and the cut lamina, for example, by placing bone chips obtained from the neck during the laminoplasty around the device. However, it must be appreciated that any spinal operation carries a risk and while we have been very confident that the present invention will offer an advantage and will not carry any greater risk than other posterior approach to the cervical spine, the design of the devices has been adopted to minimise or eliminate slippage of the device once its fitted and to function so that it will retain the enlarged spinal canal. It is considered that the present invention and the technique of applying the device represents a significant improvement in an area which is medically recognised as requiring particular care because of the consequences that follow from damaging the spinal cord.

While the devices are described above with reference to a laminoplasty procedure, they could be modified for use in other procedures. By way of example, the devices could be used to stabilise across a cut in a lamina in the thoracic region of the spine after a procedure in which a tumour is removed, or could be used to stabilise across a cut in a lamina in the lumbar region of the spine after a repair to a lumbar vertebrae, say after an operation to address lumbar stenosis.

The foregoing describes the invention including a preferred thereof so alterations and modifications as will be obvious to those skilled in the art are intended to be incorporated within the scope hereof as defined in the accompanying claims.

1. A surgical implant device for stabilising a lamina of the spine after a laminoplasty in which a cut is made in the lamina to form two lamina portions, the device comprising a longitudinally extending spacer component adapted to be fixed to the lamina across the cut, and comprising one or more members extending from one side of the spacer component to fit in the space between the two lamina portions when the device is fixed in position.

2. A surgical implant device as claimed in claim 1, wherein the spacer component is contoured to substantially conform to the surface shapes of the lamina portions.

3. A surgical implant device as claimed in claim 1, wherein the spacer component has an enlarged end configured to engage a portion of the lamina in use to distribute load.

4. A surgical implant device as claimed in claim 3, wherein the spacer component is generally L-shaped.

5. A surgical implant device as claimed in claim 1, wherein the or each member which extends from one side of the spacer component comprises one or more apertures, to enhance bone growth and assist in fusing of the lamina to the device.

6. A surgical implant device as claimed in claim 1, wherein the or each member which extends from one side of the spacer body comprises two spaced apart tabs, against which the cut surfaces of the lamina portions can abut in use.

7. A surgical implant device as claimed in claim 1, wherein the device is configured to be fixed to both lamina portions.

8. A surgical implant device as claimed in claim 1, wherein the device is configured to be fixed to the or each lamina portion by one or more fasteners passing through apertures provided in the spacer component and into the or each lamina portion.

9. A surgical implant device as claimed in claim 8, wherein the device is configured such that the head of the fastener(s) is/are accessible from the opposite side of the spacer component to that which engages the lamina portions.

10. A surgical implant device as claimed in claim 8, comprising a tab associated with each aperture which can be bent over the head of a respective fastener once tightened, to assist in maintaining the fastener in the tightened position.

11. A surgical implant device for stabilising a lamina of the spine after a laminoplasty in which a cut is made in the lamina to form two lamina portions, the device comprising a longitudinally extending spacer component adapted to be fixed to the lamina across the cut, the spacer component being contoured to conform substantially to the surface shapes of the lamina portions, and comprising one or more members extending from one side of the spacer component to fit in the space between the two lamina portions when the device is fixed in position and which comprise apertures to assist in bone growth and thereby fusing of the lamina to the device.

12. A surgical implant device for stabilizing a lamina of the spine after a procedure in which a cut is made in the lamina to form two lamina portions, the device comprising first and second spacer parts adapted to be positioned on opposite sides of the cut and connectable together to fix the device in position.

13. A surgical implant device as claimed in claim 12, wherein the first spacer part comprises a body which is arranged to extend across the cut formed in the lamina.

14. A surgical implant device as claimed in claim 13, wherein the body is contoured to conform substantially to the surface shapes of the lamina portions on the outside of the lamina.

15. A surgical implant device as claimed in claim 12, wherein the body has an enlarged end configured to engage a portion of the lamina in use to distribute load.

16. A surgical implant device as claimed in claim 15, wherein the body is generally L-shaped.

17. A surgical implant device as claimed in claim 12, wherein the second spacer part comprises a body which is arranged to extend across the cut formed in the lamina.

18. A surgical implant device as claimed in claim 17, wherein the body of the second spacer part is contoured to conform substantially to the surface shapes of the lamina portions on the inside of the lamina.

19. A surgical implant device as claimed in claim 12, wherein at least one of the spacer parts comprises one or more portions arranged to bite into the bone material of the lamina to assist in securely locating the device relative to the lamina portions.

20. A surgical implant device as claimed in claim 19, wherein both spacer parts comprise spikes depending therefrom which are arranged to bite into the bone material of the lamina to assist in fusing of the lamina to the device.

21. A surgical implant device as claimed in claim 20, wherein the spikes are positioned at or adjacent the ends of the spacer parts.

22. A surgical implant device as claimed in claim 12, wherein at least one of the spacer parts comprises one or more members extending from one side of the spacer part to fit in the space between the lamina portions when the device is fixed in position.

23. A surgical implant device as claimed in claim 12, wherein the spacer parts are configured to be connected together by a fastener extending from one spacer part and attaching to the other spacer part.

24. A surgical implant device as claimed in claim 23, wherein the device is configured such that a head of the fastener is accessible from the opposite side of the first spacer part to that which engages the lamina portions.

25. A surgical implant device as claimed in claim 23, comprising a tab associated with the aperture which can be bent over the head of the fastener once tightened, to assist in maintaining the fastener in the tightened position.

26. A surgical implant device as claimed in claim 12, wherein the lamina is in the cervical region of the spine.

27. A surgical implant device as claimed in claim 1, wherein the lamina is in the lumbar region of the spine or the thoracic region of the spine.

28. A surgical implant device for stabilizing a lamina of the spine after a laminoplasty in which a cut is made in the lamina to form two lamina portions, the device comprising first and second spacer parts adapted to be positioned on opposite sides of the cut and arranged to extend across the cut from the first lamina portion to the second lamina portion and connectable together to fix the device in position, each spacer part being contoured to conform substantially to the surface shapes of the lamina portions, and each spacer part being provided with dependent spikes which are configured to bite into the surfaces of the lamina portions in use to assist in fusing of the lamina portions to the device.

29. A method of stabilising a lamina of the spine after a procedure in which a cut is made in the lamina to form two lamina portions, comprising the steps of positioning and fixing a surgical implant device as claimed in any one of the preceding claims to the lamina portions across the cut in the lamina.

30. A method as claimed in claim 29, wherein the surgical implant device is positioned such that part of the device extends between the lamina portions to maintain the lamina portions in a spaced apart configuration.

31. A method as claimed in claim 29, wherein the lamina is in the cervical region of the spine.

32. A method as claimed in claim 31, wherein the procedure is a laminoplasty.

33. A method as claimed in claim 29, wherein the lamina is in the thoracic or lumbar region of the spine.
